(19) United States
(12) Patent Application Publication
(10) Pub. No.: US 2026/0183141 A1
(43) Pub. Date: Jul. 2, 2026

(54) PERSONAL THERMAL MANAGEMENT SYSTEM WITH COOLING OR WARMING CAPABILITY

(71) Applicant: The Science and Engineering Corps LLC, California, KY (US)

(72) Inventors: Matthew B. Sunday, California, KY (US); Joseph H Althaus, Yellow Springs, OH (US); Michael C. Moulton, Waynesville, OH (US); Thomas Mitchell, Centerville, OH (US)

(21) Appl. No.: 19/434,038

(22) Filed: Dec. 29, 2025

Related U.S. Application Data
(60) Provisional application No. 63/739,299, filed on Dec. 27, 2024.

(51) Int. Cl. A61F 7/00; A45F 3/16; A61F 7/02

(52) U.S. Cl. CPC A61F 7/0053; A45F 3/166; A61F 7/02; A61F 2007/0054; A61F 2007/0095; A61F 2007/0244

(57) ABSTRACT

A wearable thermal management system for regulating body temperature of a wearer. The system includes a reservoir holding a heat-transfer liquid, a thermal exchange pad positioned adjacent the wearer, and a pump fluidly coupled to the reservoir and the thermal exchange pad to circulate the liquid through a closed loop. A controller operates the pump in a non-continuous pattern, based on pre-programmed profiles or real-time temperature sensor feedback from the thermal exchange pad, reservoirs, environment, or wearer's skin. This intelligent control optimizes thermal exchange efficiency, power consumption of the power source, and prevents over-regulation. The system includes a hydration reservoir and a thermoregulation reservoir arranged for thermal energy transfer. The system is compact, wearable (e.g., integrated into a backpack), and adaptable for various human and non-human applications, enhancing comfort and safety.

### CROSS REFERENCE TO RELATED APPLICATIONS

The present application claims the benefit of U.S. Provisional Ser. No. 63/739,299 filed on Dec. 27, 2024, the contents of which are incorporated herein by reference in their entirety.

### GOVERNMENT RIGHTS IN INVENTION

This invention was made with an award from the Kentucky Cabinet for Economic Development, under Grant Agreement CED No. 041-2023-001-009.

### TECHNICAL FIELD

The present disclosure relates generally to personal thermal management systems. In particular, the present disclosure relates to a wearable thermal management system for regulating body temperature of a wearer through cooling or warming capabilities.

### BACKGROUND

Personal hydration systems have been developed to allow users to drink while engaged in physical activities. Such systems often include a liquid reservoir that a user carries in a backpack, waist pack, vest, or other body mounted equipment management system, and a drinking tube that extends from the liquid reservoir to allow the user to drink from the tube. The drinking tube may have a bite valve at the end that allows the user to start the flow of liquid by biting on the valve. Personal hydration systems of the type described above generally do not provide the ability to cool or warm the outside surfaces of a wearer's body.

Numerous attempts have been made to develop personal cooling systems. Often such efforts have involved creating a garment, such as a vest, and filling the garment with ice, or some other cooling substance. Garments have also been developed that circulate a cooling fluid through the garment using integrated tubing.

Cooling systems of the type described above typically suffer several drawbacks. One drawback is that providing a wearer with an additional garment to wear in order to cool themselves is an inherent disadvantage since it requires the user to add an additional layer of clothing when they may already be operating under hot conditions. Another drawback of many such garments is that after a period of time the cooling medium will become warmer and lose its ability to cool the wearer. When this happens, in some cases, the wearer may either have to continue to wear the garment, carry the garment, or discard the garment.

### SUMMARY

The present disclosure relates to a wearable thermal management system with cooling or warming capability configured to regulate the body temperature of a wearer. The system includes a reservoir or bladder configured to hold a heat-transfer medium, a thermal exchange pad positioned adjacent to the wearer, and a pump fluidly coupled to the reservoir and the thermal exchange pad to circulate the heat-transfer medium in a closed loop via a plurality of conduits. The reservoir may be integrated with specific compartments, such as a distribution manifold pocket and a pump pocket, to manage fluid distribution and secure the pump. A controller is operatively coupled to the pump to manage the flow of the heat-transfer medium, where the controller may be configured to operate the pump in a non-continuous pattern or duty cycle pattern. This operation may be based on pre-programmed profiles or real-time feedback from temperature sensors located at the thermal exchange pad to optimize thermal exchange efficiency and power consumption. Further, the system may utilize a potable medium to provide both hydration and thermal regulation from a single source, and is designed to be lightweight, compact, and easily integrable into wearable items such as backpacks.

In an aspect, the present disclosure relates to a wearable thermal management system. The system may include a reservoir holding a heat-transfer liquid and a thermal exchange pad positioned adjacent a wearer. The system may include a pump fluidly coupled to the reservoir and the thermal exchange pad to circulate the heat-transfer liquid through a closed loop Further, the system may include a controller operatively coupled to the pump, where the controller regulates volumetric flow of the heat-transfer liquid through at least a portion of the thermal exchange pad to control thermal transfer between the wearer and the heat-transfer liquid

In an embodiment, the controller may operate the pump in a substantially continuous mode during use.

In an embodiment, the controller may operate the pump in an intermittent mode.

In an embodiment, the controller may vary a flow rate of the pump while maintaining continuous operation.

In an embodiment, the system may include a bypass conduit configured to selectively divert a portion of the heat-transfer liquid around at least a portion of the thermal exchange pad.

In an embodiment, thermal transfer may be further regulated by modulating hydraulic resistance within the thermal exchange pad or within the closed loop.

In an embodiment, the system may include a memory storing a first pump operation profile that defines operation of the pump.

In an embodiment, the pump operation profile may include a predetermined pump on-time and pump off-time.

In an embodiment, the memory stores a plurality of pump operation profiles including the first pump operation profile, from which the wearer selects directly or indirectly the first pump operation profile.

In an embodiment, a second pump operation profile of the plurality of pump operation profiles defines continuous pump activation.

In an embodiment, a third pump operation profile of the plurality of pump operation profiles may include a duty-cycle pattern.

In an embodiment, the system may include one or more temperature sensors to detect any or a combination of temperatures associated with the system, wearer of said system, and operating environment, and where the controller regulates operation of the pump or volumetric flow based at least in part on the detected temperatures.

In an embodiment, the one or more temperature sensors are positioned to detect the temperature of any or a combination of heat-transfer liquid entering and/or exiting the thermal exchange pad, temperature associated with the reservoir, and temperature associated with the pump.

In an embodiment, the controller increases frequency of pump activation as the temperature of the heat-transfer liquid increases.

In an embodiment, the controller increases frequency of pump activation as the temperature of the heat-transfer liquid decreases.

In an embodiment, the controller decreases frequency of pump activation when the temperature of the heat-transfer liquid is below a first predetermined threshold.

In an embodiment, the controller decreases frequency of pump activation when the temperature of the heat-transfer liquid is above a second predetermined threshold.

In an embodiment, the controller automatically adjusts the volumetric flow based on a thermal model stored in a memory.

In an embodiment, the system may include a flow control element operatively coupled to the pump and positioned within the closed loop, and where the controller regulates a flow rate of the heat-transfer liquid through the thermal exchange pad by modulating the flow control element independently of pump on/off operation.

Another embodiment of the present disclosure pertains to a wearable thermal management system. The system may include a reservoir holding a heat-transfer liquid and a thermal exchange pad positioned adjacent a wearer. Further, the system may include a pump fluidly coupled to the reservoir and the thermal exchange pad to circulate the heat-transfer liquid through a closed loop and a non-electronic fluid circulation mechanism operatively coupled to the pump or the closed loop to regulate volumetric flow of the heat-transfer liquid without digital or programmable electronic control.

In an embodiment, the fluid circulation mechanism may include any or a combination of cam configured to intermittently depress an actuator of the pump, manual crank, squeeze bulb, and self-pumping upon movement of the wearer.

In an embodiment, the fluid circulation mechanism may include a spring-loaded escapement that releases periodically to activate the pump.

In an embodiment, the fluid circulation mechanism may include a bimetallic switch that interrupts electrical power based on temperature.

In an embodiment, the fluid circulation mechanism may include an analog RC timing circuit configured to intermittently energize the pump.

In an embodiment, the fluid circulation mechanism may include an electromechanical relay driven by a periodic timing signal.

In an embodiment, the system is integrated into a backpack so as to route tubing and wiring internally, and where the pump is any or a combination of a diaphragm pump, a peristaltic pump, and a centrifugal pump.

In an embodiment, operation of the system is selected so as to maintain a substantially constant thermal sensation at the wearer.

Another embodiment of the present disclosure pertains to a wearable thermal management system that may include a hydration reservoir to hold a drinkable liquid, where the hydration reservoir being operatively coupled with a drinking tube. The system may include a thermoregulation reservoir to hold a heat-transfer liquid where the thermoregulation reservoir being operatively coupled to a pump that circulates the heat-transfer liquid through a thermal exchange pad positioned adjacent to a wearer so as to form a closed thermal loop. Further, the hydration reservoir and the thermoregulation reservoir are arranged so as to allow thermal energy transfer through a thermal interface so as to enable the thermoregulation reservoir to absorb heat or cold from the hydration reservoir.

In an embodiment, the hydration reservoir and the thermoregulation reservoir form partitioned sections of a single main reservoir.

In an embodiment, the hydration reservoir and the thermoregulation reservoir are sections that are operatively coupled with each other.

In an embodiment, the system may include an input/output connector operatively coupled with the pump and a distribution manifold connector operatively coupled with the pump on one end and the thermal exchange pad on the other end.

In an embodiment, the thermal exchange pad acts as a cooling pad if the heat-transfer liquid is cold, and acts as a heating pad if the heat-transfer liquid is hot/warm.

In an embodiment, the thermal exchange pad may include an inlet medium temperature sensor and/or an outlet medium temperature sensor to measure a temperature of the heat-transfer liquid entering and exiting the thermal exchange pad, based on which an amount of heat extracted by the thermal exchange pad is determined, and wherein circulation of the heat-transfer liquid is regulated by controlling volumetric flow through the thermal exchange pad.

In an embodiment, the system may include one or more temperature sensors operatively coupled with any or a combination of the thermoregulation reservoir, the pump, the thermal exchange pad, and the wearer, based on which, amount of heat extracted is determined and circulation of the heat-transfer liquid is regulated by controlling volumetric flow. In an embodiment, the thermal exchange pad comprises a first layer and a second layer, and a baffle that runs between the said layers to keep the layers in contact with each other.

In an embodiment, the system may include a backpack operatively coupled with the thermal exchange pad.

### BRIEF DESCRIPTION OF THE DRAWINGS

While the specification concludes with claims which particularly point out and distinctly claim this technology, it is believed this technology will be better understood from the following description of certain examples taken in conjunction with the accompanying drawings, in which like reference numerals identify the same elements and in which:

FIG. 1 shows one version of a wearable thermal management system, in accordance with embodiments of the present disclosure.

FIG. 2 shows one version of a reservoir of the wearable thermal management system, in accordance with embodiments of the present disclosure.

FIG. 3 shows a first side of a thermal exchange pad of the wearable thermal management system, in accordance with embodiments of the present disclosure.

FIG. 4 shows a second side of a thermal exchange pad of the wearable thermal management system, in accordance with embodiments of the present disclosure.

FIG. 5 shows a front side view of a backpack utilizable as a component of the wearable thermal management system, in accordance with embodiments of the present disclosure.

FIG. 6 shows the reservoir of FIG. 2 located within an interior of the backpack of the wearable thermal management system, in accordance with embodiments of the present disclosure.

FIG. 7 shows an open top pocket of the backpack of the wearable thermal management system, in accordance with embodiments of the present disclosure.

FIG. 8 shows an open bottom pocket of the backpack of the wearable thermal management system, in accordance with embodiments of the present disclosure.

FIG. 9 shows a back side view of the backpack of the wearable thermal management system, in accordance with embodiments of the present disclosure.

FIG. 10 shows the thermal exchange pad of FIG. 3 in a secured position against the backpack of the wearable thermal management system, in accordance with embodiments of the present disclosure.

FIG. 11 shows an electronics system utilizable as a component of the wearable thermal management system, in accordance with embodiments of the present disclosure.

The drawings are not intended to be limiting in any way, and it is contemplated that various embodiments of the technology may be carried out in a variety of other ways, including those not necessarily depicted in the drawings. The accompanying drawings incorporated in and forming a part of the specification illustrate several aspects of the present technology, and together with the description serve to explain the principles of the technology; it being understood, however, that this technology is not limited to the precise arrangements shown.

### DETAILED DESCRIPTION

The following description of certain examples of the technology should not be used to limit its scope. Other examples, features, aspects, embodiments, and advantages of the technology will become apparent to those skilled in the art from the following description, which is by way of illustration, one of the best modes contemplated for carrying out the technology. As will be realized, the technology described herein is capable of other different and obvious aspects, all without departing from the technology. Accordingly, the drawings and descriptions should be regarded as illustrative in nature and not restrictive.

It is further understood that any one or more of the teachings, expressions, embodiments, examples, etc. described herein may be combined with any one or more of the other teachings, expressions, embodiments, examples, etc. that are described herein. The following-described teachings, expressions, embodiments, examples, etc. should therefore not be viewed in isolation relative to each other. Various suitable ways in which the teachings herein may be combined will be readily apparent to those of ordinary skill in the art in view of the teachings herein. Such modifications and variations are intended to be included within the scope of the claims.

For clarity of disclosure, the terms “upper end” and “lower end” are defined herein relative to a human/mammalian wearer of the thermal management system. The term “upper end” refers to the position of an element closer to the head of the human/mammalian wearer of the thermal management system. The term “lower end” refers to the position of an element closer to the lower extremities of the human/mammalian wearer of the thermal management system.

The present invention relates generally to personal (wearable) thermal management systems, and more particularly to thermal management systems with cooling and/or warming capabilities, and the components thereof. The term “personal” refers to humans and other mammals alike. The disclosure can alternatively be thought of as a wearable thermal management system. The terms “temperature adjustment capability” or “body temperature adjustment capability” may he used herein to refer to a system that may be capable of providing cooling and/or warming capability.

The personal thermal management systems of the present disclosure are portable, and thus, the entire system may be capable of being worn by a human/mammal. The personal thermal management systems described herein may be suitable for soldiers, law enforcement officers, firefighters, hikers, bicycle riders, motorcyclists, confined space workers, those who deal with thermoregulatory challenges such as geriatrics or medically compromised individuals, or any occupation, activity, or situation wherein personal thermal management would be considered beneficial.

A primary object of the present disclosure is to provide a wearable thermal management system for regulating the body temperature of a user through cooling or warming capabilities, thereby enhancing comfort and safety in varying environmental conditions. Another objective is to utilize a single reservoir for both hydration and thermal regulation, eliminating the need for separate non-consumable fluids and reducing the overall weight carried by the user. It is also an object to provide a system that optimizes power consumption and thermal efficiency by operating a pump in a non-continuous duty cycle based on real-time sensor feedback from the wearer, the medium, and the environment. Furthermore, the disclosure aims to provide a compact, low-profile system integrated into wearable gear like backpacks, ensuring seamless operation without impeding user mobility while preventing over-cooling or over-heating through intelligent monitoring.

The present disclosure relates to a wearable thermal management system with cooling or warming capability configured to regulate the body temperature of a wearer. The system includes a reservoir or bladder configured to hold a heat-transfer medium, a thermal exchange pad positioned adjacent to the wearer, and a pump fluidly coupled to the reservoir and the thermal exchange pad to circulate the heat-transfer medium in a closed loop via a plurality of conduits. The reservoir may be integrated with specific compartments, such as a distribution manifold pocket and a pump pocket, to manage fluid distribution and secure the pump. A controller is operatively coupled to the pump to manage the flow of the heat-transfer medium, where the controller may be configured to operate the pump in a non-continuous pattern or duty cycle. This operation may be based on pre-programmed profiles or real-time feedback from temperature sensors located at the thermal exchange pad to optimize thermal exchange efficiency and power consumption. Further, the system may utilize a potable medium to provide both hydration and thermal regulation from a single source, and is designed to be lightweight, compact, and easily integrable into wearable items such as backpacks.

In an aspect, the present disclosure relates to a wearable thermal management system. The system may include a reservoir holding a heat-transfer liquid and a thermal exchange pad positioned adjacent a wearer. Further, the system may include a pump fluidly coupled to the reservoir and the thermal exchange pad to circulate the heat-transfer liquid through a closed loop, were the pump being operated by a controller in a non-continuous pattern.

Another embodiment of the present disclosure pertains to a wearable thermal management system. The system may include a reservoir holding a heat-transfer liquid and a thermal exchange pad positioned adjacent a wearer. Further, the system may include a pump fluidly coupled to the reservoir and the thermal exchange pad to circulate the heat-transfer liquid through a closed loop and a non-electronic fluid circulation mechanism to operate the pump in a non-continuous pattern.

Another embodiment of the present disclosure pertains to a wearable thermal management system that may include a hydration reservoir to hold a drinkable liquid, where the hydration reservoir being operatively coupled with a drinking tube. The system may include a thermoregulation reservoir to hold a heat-transfer liquid where the thermoregulation reservoir being operatively coupled to a pump that circulates the heat-transfer liquid through a thermal exchange pad positioned adjacent to a wearer so as to form a closed thermal loop. Further, the hydration reservoir and the thermoregulation reservoir are arranged so as to allow thermal energy transfer through a thermal interface so as to enable the thermoregulation reservoir to absorb heat or cold from the hydration reservoir.

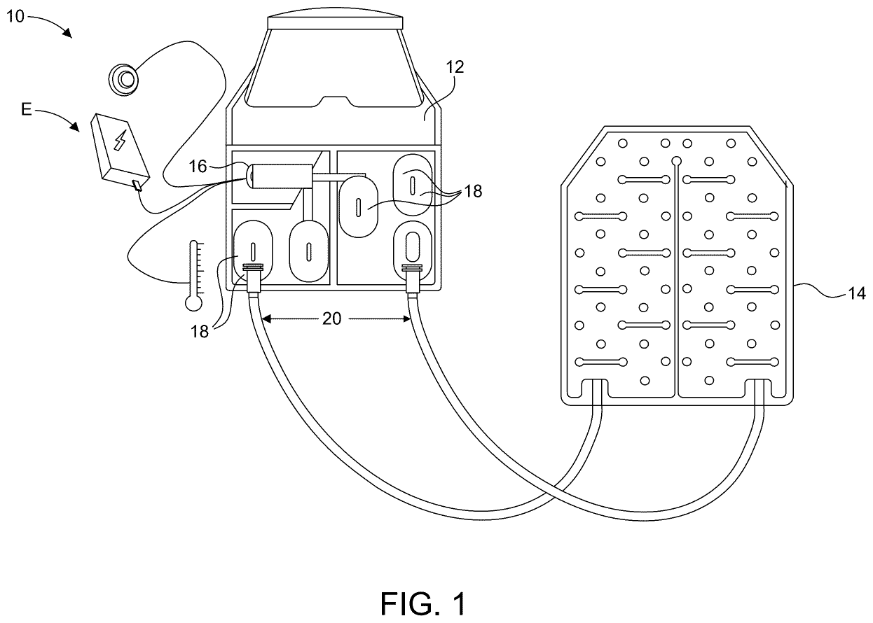

Various embodiments with respect to the present disclosure will be explained in detail with reference to FIGS. 1-11 .

FIG. 1 shows one version of a wearable thermal management system 10 having cooling and/or warming capability. The system 10 can include a reservoir/liquid reservoir/bladder 12, a thermal exchange pad 14 configured for wearing adjacent a wearer's body, a pump 16 configured for generating a flow of a medium, such as a heat transfer liquid, a plurality of ports 18 (discussed in detail below), and a plurality of conduits/tubing 20 to control the path of the medium moving based on the flow of the medium generated by the pump 16. At least some ports 18 and conduits 20 may be configured to be arranged in a closed loop configuration when joined to the reservoir 12 and the thermal exchange pad 14. The thermal exchange 14 may be configured as a layer, system or subsystem or any other configuration and/or construction that can perform equivalent functions. The thermal exchange pad 14 may be considered to act as either a cooling pad 14, if the medium within reservoir 12 may be cold, or a heating pad 14, if the medium within the reservoir 12 may be hot/warm. In one or more versions, the pump 16 may be bidirectional, which enhances functionality. Functionality may be enhanced with the pump 16 being bidirectional by allowing for the ability to draw a medium into the system 10, purge the system, and to leverage the manifold (discussed below) in other ways.

It should be understood that in some versions, the present disclosure may relate to the entire wearable thermal management system 10, including the reservoir 12 and thermal exchange pad 14. In one or more embodiments, it may be contemplated that reservoir 12 and thermal exchange pad 14 are combined into one unified system, as long as reservoir 12 is thermally insulated from thermal exchange pad 14. In other embodiments, the present disclosure may relate to certain components thereof (such as the manifold system (discussed in detail below) or the control system (discussed in detail below)) and may not require that reservoir 12 and/or thermal exchange pad 14 be a part thereof.

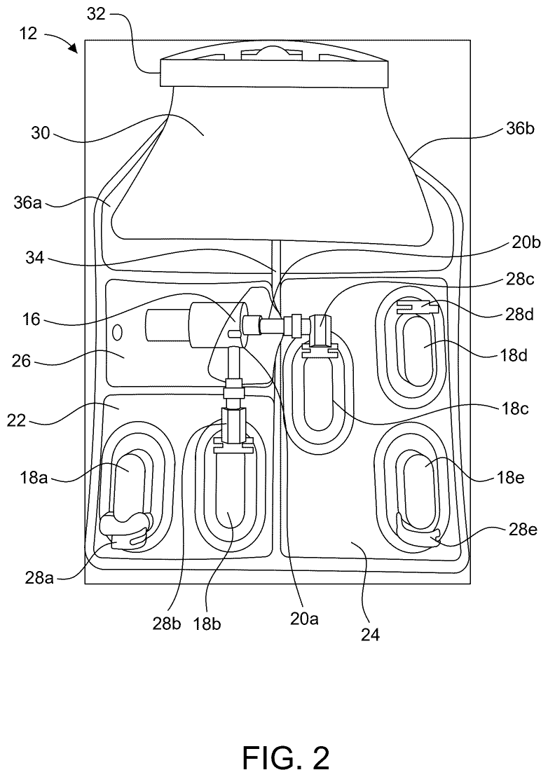

As shown on its own in FIG. 2, reservoir 12 may be any suitable type of container capable of holding a heating/cooling medium, and that may be portable and wearable by a user. In one or more embodiments, the term user contemplates that system 10 may be utilized by both humans and animals of any kind and in any environment from land to sea and in both extremely warm and extremely cold environments. The reservoir 12 may be closed and thermally insulated (or to be provided with insulating material joined thereto) in order to maintain the temperature of the medium contained therein. In other cases, even if the reservoir 12 is not thermally insulated, the reservoir 12 may be provided inside an insulated pocket. In one or more versions, the reservoir 12 may be a commercially available medium reservoir that may be of a standard type. In an example the reservoir 12 may be a Chemical, Biological, Radiological, and Nuclear (CBRN) medium reservoir. For example, the reservoir 12 may include a commercially available hydration reservoir, and can be used with the same supporting accessory that may be designed to carry the reservoir 12, such as a backpack, waist pack, vest, or other body mounted equipment management system.

In one or more versions, the reservoir 12 may be integrated with an inlet/outlet pocket 22, a distribution manifold pocket 24, and a pump pocket 26. Inlet/outlet pocket 22 provides direct access into the reservoir 12, while the distribution manifold pocket 24 provides a separate reservoir for medium that may be separate and distinct from the reservoir 12. The Pump pocket 26 provides an enclosure to hold pump 16. Located on inlet/outlet pocket 22 are two access ports 18a, 18b bonded onto the inlet/outlet pocket 22. In one or more versions, access ports 18a, 18b have been RF welded onto the inlet/outlet pocket 22. Contained with access port 18a may be a connector 28a which may act as in inlet for medium traveling from the thermal exchange pad 14 to the reservoir 12. In one or more versions, the connector 28a may or may not include a valve. Contained within access port 18b may be a connector 28b that may act as an outlet for medium traveling from the reservoir 12, to the thermal exchange pad 14. In one or more versions, connector 28b may or may not include a valve.

The distribution manifold pocket 24 contains three access ports 18c, 18d, and 18e bonded onto the distribution manifold pocket 24. In one or more versions, access ports 18c, 18d, and 18e have been RF welded onto the distribution manifold pocket 24. Contained within access port 18c may be a connector 28c which may act as an inlet for medium traveling from the reservoir 12 into the distribution manifold pocket 24. In one or more versions, connector 28c may or may not include a valve. Contained within access port 18e may be a connector 28e which may act as an outlet for medium leaving the distribution manifold pocket 24 and traveling into the thermal exchange pad 14. In one or more versions, connector 28e may or may not include a valve. Access port 18d may be optional, however, if present, contained within access port 18d may be a connector 28d which may act as an outlet for medium traveling from the distribution manifold pocket 24 into an optional drinking tube port or medium distribution device (not shown). In one or more versions, the connector 28d may or may not include a valve. In one or more versions, if optional drinking tube port or medium distribution device (not shown) is present, then it may include a filter of some sort to make sure medium exiting optional drinking tube port or medium distribution device can be consumed alternatively all involved components may be constructed from food-safe/food-grade materials and the working medium may be potable drinking water or a nutrient drink. The pump 16 may be connected to the access port 18b through tubing 20a while the pump 16 may be connected to the access port 18c through tubing 20b.

In one or more versions, when the pump 16 has been activated, the pump 16 may draw medium from the reservoir 12 through the access port 18b. The medium may travel through the tubing 20a, through the pump 16, through the tubing 20b and through the access port 18c. The medium can then be held in the reservoir of the distribution manifold pocket 24. That medium may travel through the access port 18e, through the tubing 20c, and enter into the thermal exchange pad 14. Additionally, if the access port 18d is present, medium held in the reservoir 12 of the distribution manifold pocket 24 may also travel through access port 18d, through tubing (not shown), and into the optional drinking tube port or medium distribution device (not shown). Once the medium travels through the thermal exchange pad 14 (as discussed in detail below), the medium moves through tubing 20e, through access port 18a, and back into the reservoir 12.

In one or more versions, the reservoir 12 additionally includes a funnel shaped fill neck 30, which provides access to the reservoir 12 to clean or fill the reservoir 12 with a medium by which to keep the medium cold or hot, such as ice, hot/cold packs, etc. Fill neck 30 may be sealable, and in one or more versions, a closure device 32 may be used to close off access to the reservoir 12 such that the medium within the reservoir 12 cannot escape the reservoir 12 through fill neck 30. In one or more versions, the closure device 32 may be a slidable sealer, a clip, a cam system, a zip-lock type system, or combinations thereof.

In one or more versions, the reservoir 12 additionally includes a baffle 34 that runs between a front wall 36a and a rear wall 36b of the reservoir 12. Baffle 34 allows front wall 36a and rear wall 36b to stay in contact with one another along the length of baffle 34. This close contact of front wall 36a and rear wall 36b allows the reservoir 12 to remain in somewhat of a compact state, which may allow for the reservoir 12 to have a low profile to comfortably fit within a backpack, as discussed in detail below.

In one or more versions, the reservoir 12 can contain any medium suitable for cooling and/or heating the wearer's body. Suitable mediums include but are not limited to liquids such as water in liquid form (or some of which may be in the form of ice). Such mediums may include non-consumable liquids, and gases, such as air. In one or more versions, it may be desirable for the medium to be water so that it may be capable of being consumed by the wearer and/or used for other purposes as described herein. Having the same medium serve a cooling or heating function as well as a hydration function eliminates the need for the wearer to carry a separate reservoir of a heat transfer medium (such as polyethylene glycol) that cannot be consumed by the wearer. The reservoir 12 can contain any suitable amount of medium. For example, the reservoir 12 can contain about 1 to about 3 Liters of a liquid medium. The holding capacity of the reservoir 12 may be greater than that of the thermal exchange pad 14 so that one or more times the amount of medium may be pumped to the thermal exchange pad 14 to heat or cool the wearer, and then recirculated back to the reservoir 12 where such recirculated medium may be mixed with the medium in the reservoir 12. In some cases, the holding capacity of the reservoir 12 may be 10%, 20%, 30%, . . . up to 500% or more, greater than that of the thermal exchange pad 14.

The medium in the reservoir 12 may be cooled or heated to any suitable temperature. For instance, the medium in the reservoir 12 may be cooled or heated so that the medium contains at least some medium that may be at an initial temperature that differs from ambient temperature where personal thermal management system 10 may be worn. If the medium is water, it may for example, be chilled to a temperature of between about 35-50° F. The medium in the reservoir 12 can be cooled by refrigerating the medium prior to adding it to the reservoir 12, or by adding ice to the reservoir 12. Alternatively, reservoir 12 with the medium therein can be refrigerated before a user may wear it. If reservoir 12 contains ice, sufficient medium should be present at the time needed for cooling the wearer's body in order to permit circulation from reservoir 12 to thermal exchange pad 14. If desired, system 10 may be used as a wearable thermal management system to warm the wearer's body, then the medium can be heated before being added to the reservoir 12.

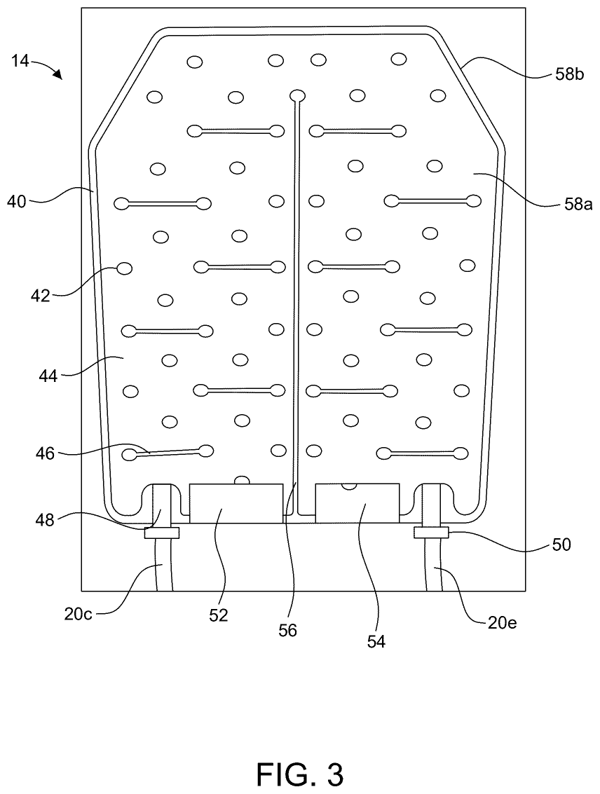

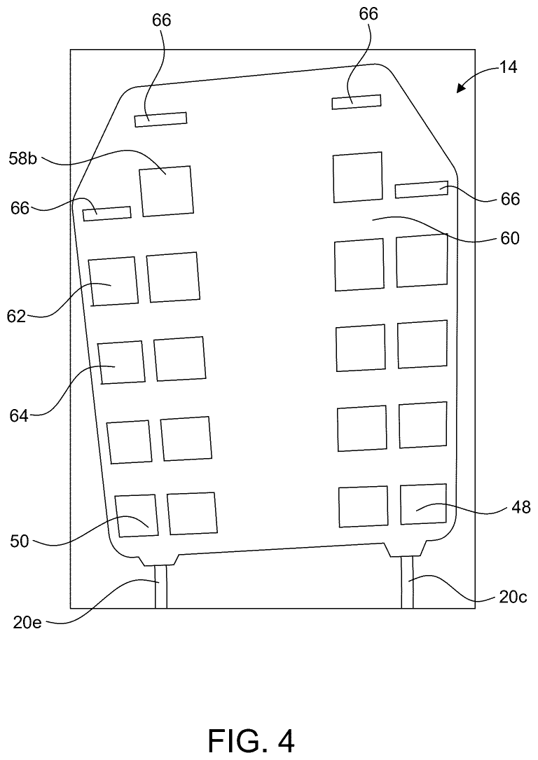

As shown on its own in FIG. 3 and FIG. 4, the thermal exchange pad 14 for wearing adjacent to a wearer's body can include any suitable type of cooling and/or heating pad that provides for the circulation of a cooling or heating medium therethrough. The thermal exchange pad 14 may be thermally conductive so that it allows the medium to either thermally cool or heat the user, depending on the temperature of the medium. The thermal exchange pad 14 may include from two to three layers of material (typically flexible material such as film) that are bonded together adjacent to their periphery to provide a relatively flat sealed compartment with a tight perimeter seal 40. In one or more versions, the term bonded includes one or more of RF welding, molding, and/or gluing. The thermal exchange pad 14 may be bonded together at various bond locations 42 that are inside of the periphery to provide internal flow channels 44 for directing the flow of medium through the thermal exchange pad 14. Spot bonds 46 can also be provided to hold the layers of the thermal exchange pad 14 together and optionally to add turbulence to the flow of medium through the thermal exchange pad 14. The thermal exchange pad 14 may have an inlet opening 48 which allows medium to enter the interior of the thermal exchange pad 14 via tubing 20c and an outlet opening 50 that allows medium to exit the thermal exchange pad 14 and return to the reservoir 12 via the tubing 20e.

In one or more versions, the thermal exchange pad 14 also includes an inlet medium temperature sensor 52 and an outlet medium temperature sensor 54. Inlet medium temperature sensor 52 and outlet medium temperature sensor 54 tracks the temperature of medium entering and exiting the thermal exchange pad 14. The system 10 may monitor the temperature of the medium when the medium enters the thermal exchange pad 14 and comparing the temperature of the medium entering the thermal exchange pad 14 to the temperature of the medium when the medium exits the thermal exchange pad 14 allows for a determination of an amount of heat extracted from the user wearing the wearable thermal management system 10. This feedback may allow to regulate circulation of the heat-transfer liquid by controlling volumetric flow on the amount of time that the personal thermal management system 10 can cycle the medium, as will be discussed in detail below. In one or more versions, the thermal exchange pad 14 may include just a singular temperature sensor that captures temperature data on the medium coming and going from the thermal exchange pad 14.

In one or more versions, the thermal exchange pad 14 also includes a baffle 56 that runs between a first layer 58a and a second layer 58b of the thermal exchange pad 14. Baffle 56 allows first layer 58a and second layer 58b to stay in contact with one another along the length of baffle 34. This close contact of first layer 58a and second layer 58b allows the thermal exchange pad 14 to remain in somewhat of a compact state.

FIG. 4 shows a rear version of the thermal exchange pad 14, which shows the thermal exchange pad 14 including an optional third layer 60 located adjacent second layer 58b. In one or more versions, if present, third layer 60 increases the durability of the thermal exchange pad 14 in regard to attaching and detaching the thermal exchange pad 14 to the wearable item, such as the backpack. Third layer 60 may be bonded along only the periphery of second layer 58b, such that there may be a space 62 between the body of third layer 60 and second layer 58b everywhere except for along the periphery where the bond may be located. In one or more versions, third layer 60 is made from a cloth material such as a durable fabric that may be compatible with processing techniques. Third layer 60 adds additional durability to the thermal exchange pad 14. Third layer 60 further includes a Pouch Attachment Ladder System (PALS) 64. PALS is a universal attachment scheme which is a grid-based system of 25 mm (1 in) wide grids with 38 mm (1.5 in) spacing between each row of grids. PALS 64 may be utilizable to secure the thermal exchange pad 14 to a wearable item, such as a backpack, an embodiment of which is discussed in detail below. Also integrated into third layer 60 are a plurality of strap loop sections 66, which may allow for a strap from the wearable item, such as the backpack, to be able to be looped back down or up once the strap has been weaved through PALS 64. In one or more versions wherein optional third layer 60 is not present, PALS 64 may be secured directly to the thermal exchange pad 14.

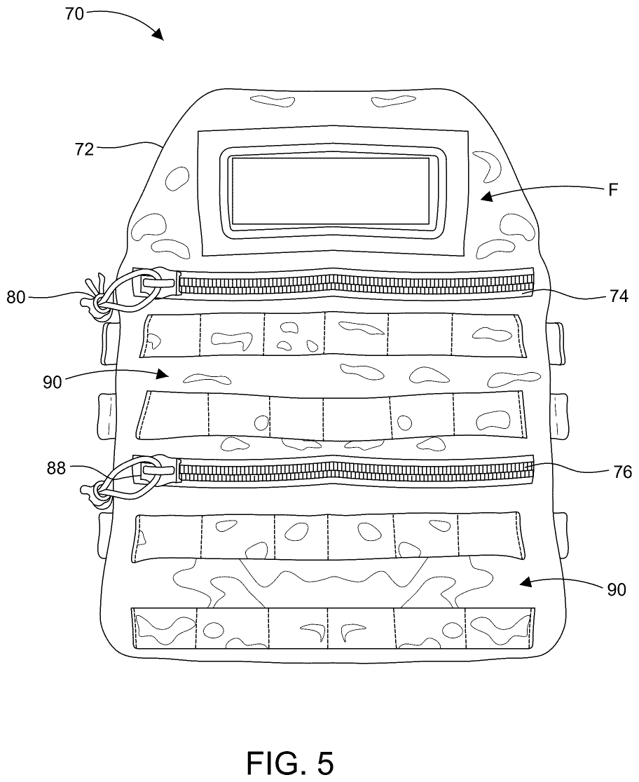

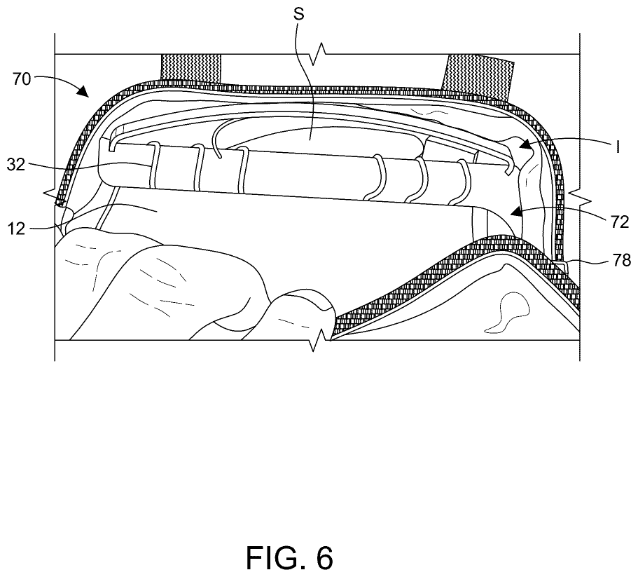

FIGS. 5 through 9 show various views of a backpack 70 utilizable as a component of the wearable thermal management system 10. As shown in FIG. 5, a front side F of backpack 70 includes a backpack opening 72 at a top end thereof, a top opening 74, and a lower opening 76. In one or more versions, backpack opening 72 provides access to an interior I of backpack 70. In one or more versions, as discussed below in more detail and as shown in FIG. 6, the reservoir 12 may be carried within interior I of the backpack 70. In one or more versions, interior I of backpack 70 further includes a central rear stiffener S that may be integrated into the backpack 70 to provide increased rigidity to the backpack 70 and for good contact for the thermal exchange pad 14 with the user. In one or more versions, backpack opening 72 can be opened and closed through the use of a securement device 78. In one or more versions, and as shown in the figures, the securement device 78 may be a zipper system. As shown, the zipper system 78 includes a first slider and a second slider to allow for backpack opening 72 to be open or sealed from either side of the backpack 70.

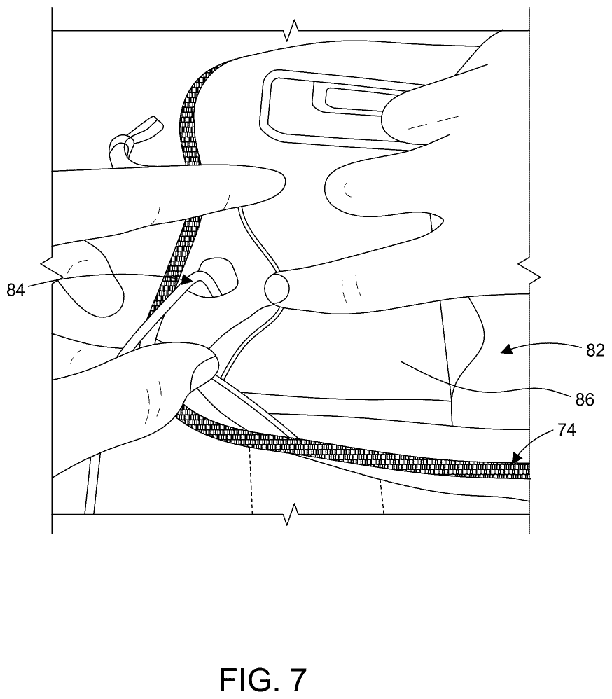

In one or more versions, top opening 74 can be opened and closed through the use of a securement device 80. In one or more versions, and as shown in the Figures, the securement device 80 may be a zipper system. In one or more versions, such as shown in FIG. 7, the top opening 74 provides access to a top pocket 82. In one or more versions, the top pocket 82 may be separate and distinct from interior I of the backpack 70, except for a small wire access hole 84, which will be discussed in detail below. In one or more versions, the top pocket 82 may be utilized to hold and keep secure an electronics system E which powers and controls pump 16 of the reservoir 12. The specific components of the electronics system E will be discussed in detail below. In one or more versions, the top pocket 82 further includes a hook and loop mount 86 utilizable as a mounting position for a Printed Circuit Board (PCB) of the electronics system E. In one or more versions, other components of electronics system E could be mounted on hook and loop mount 86. In yet other versions, PCB may be mounted at a base of the pump 16 within an environmentally protected housing. In such a version, other aspects of the electronics system E, discussed in detail below, may be housed within the top pocket 82 or 72. In such a version, various aspects of the electronics system E may also be affixed to the outside of the backpack 70.

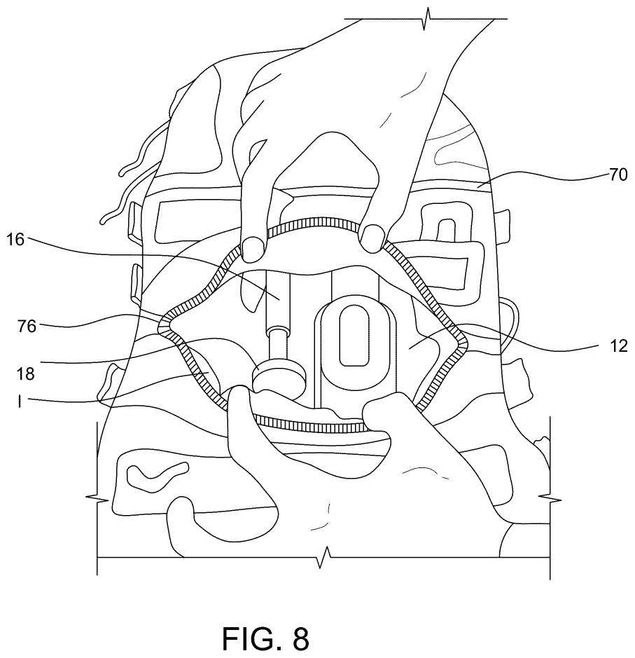

In one or more versions, bottom opening 76 can be opened and closed through the use of a securement device 88. In one or more versions, and as shown in the Figures, securement device 88 may be a zipper system. In one or more versions, such as shown in FIG. 8, the bottom opening 76 provides access to interior I of the backpack 70. In one or more versions, the bottom opening 76 may provide access to pump 16 and the plurality of ports 18 when the reservoir 12 may be contained within interior I of the backpack 70.

In one or more versions, front side F of the backpack 70 also includes a Pouch Attachment Ladder System (PALS) 90. The PALS 90, if present, may be utilized to secure other equipment to the backpack 70 such as, but not limited to radio pouches, medical kit pouches, magazine pouches, flashlights, and combinations thereof.

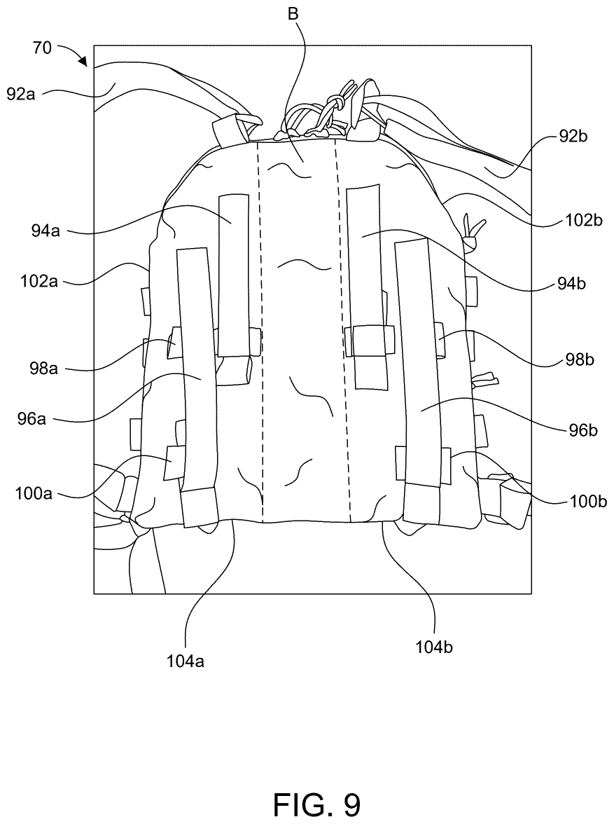

As shown in FIG. 9, a back side B of the backpack 70 includes a first shoulder strap 92a and a second shoulder strap 92b. In one or more versions, shoulder straps 92a, 92b are adjustable to allow for a user to customize the position of the backpack 70 on their shoulders. Back side B of the backpack 70 further includes a PALS strap system that may include a first interior strap connector 94a, a second interior strap connector 94b, a first exterior strap connector 96a, a second exterior strap connector 96b, a first upper strap securement device 98a, a second upper strap securement device 98b, a first lower strap securement device 100a, and a second lower strap securement device 100b. The PALS strap system may be utilized to secure PALS 64 of the thermal exchange pad 14 into position on back side B of the backpack 70.

Back side B of the backpack 70 also includes a first pass through eyelet 102a and a second pass through eyelet 102b located along an upper periphery of back side B. As discussed above, the distribution manifold pocket 24 may provide access to an optional drinking tube port or medium distribution device (not shown). If present, the optional drinking tube port, or medium distribution device could travel out of either eyelet 102a or 102b, dependent on which side of the body the user prefers having access to the optional drinking tube port or medium distribution device. Back side B of the backpack 70 also includes a first access opening window 104a and a second access opening window 104b located along a lower periphery of back side B. In one or more versions, pad tubing 20c may be connected to connector 28e and travel from the reservoir 12 located within interior I of the backpack 70, through first access opening window 104a, and arrive at the thermal exchange pad 14 and tubing 20e may travel from the thermal exchange pad 14, through second access opening window 104b, and back to the reservoir 12, located within interior I of the backpack 70, through the connector 28a.

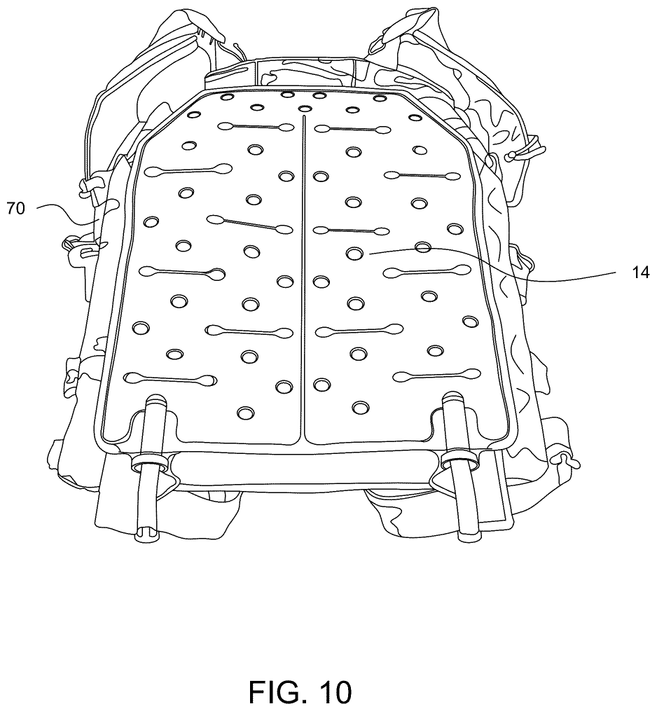

FIG. 10 shows an example version of the thermal exchange pad 14 secured to a back side B of the backpack 70. Although not shown in FIG. 10, the PALS strap system may be utilizable with PALS 64 of the thermal exchange pad 14 to position the thermal exchange pad 14 on back side B of backpack 70. Although FIG. 10 shows a version wherein the thermal exchange pad 14 may be secured directly to the backpack 70, other embodiments of system 10 are contemplated. For example, the reservoir 12 could be carried within interior I of the backpack 70, the PALS strap system could be utilized to secure an exterior surface of body armor (not shown) to the backpack 70. The thermal exchange pad 14, fluidly connected to the reservoir 12 within interior I of the backpack, could then be secured to an interior surface of body armor utilizing slots, various combinations of strap loop sections 66 and/or second layer 58b of the thermal exchange pad 14, and lengths of webbing, the strap connector 94a, strap the connector 94b, the strap connector 96a, and/or the strap connector 96b, to suspend and secure it internal to the body armor. In one or more versions, as long as there is some thermal separation and thermal gradient, between the medium within the reservoir 12 and the thermal exchange pad 14, and the thermal exchange pad 14 is positioned adjacent the user, system 10 as described herein can be utilized to heat or cool the user. With the context of the present disclosure, “adjacent the user” can mean directly against the skin of the user or can mean through an article(s) of clothing.

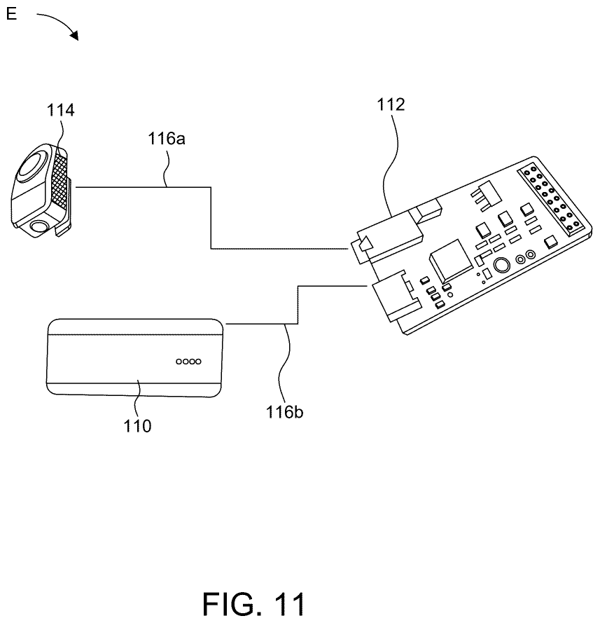

In one or more versions, the electronics system E, which powers and controls pump 16, may be stored within the top pocket 82 of the backpack 70. In one or more versions, the electronics system E, as shown in FIG. 11, includes a power source 110, a printed circuit board assembly (PCBA) 112, an On/Off mechanism 114, and optionally associated wiring 116. In one or more versions, the power source 110 may be a battery, with variable size depending on desired run time. In one or more versions, the PCBA 112 may include power management circuitry, computational circuitry, and power switching circuitry. In one or more versions, On/Off mechanism 114 may be selected from a switch, a button, or a touch sensor. In one or more versions, the components of electronics system E are connected together through wiring 116 as shown, and in yet other versions, components of electronics system E may be connected together wirelessly. Although FIG. 11 shows optional associated wiring 116, the positioning of the wiring from the power source 110, the PCBA, and 112 On/Off mechanism is not shown to scale or for the accuracy of its placement. On/Off mechanism 114 is secured to the power source 110 through wiring 116a, and the power source 110 may be secured to the pump 16 through wiring 116b.

The pump 16 circulates the heat-transfer liquid through a closed loop and is operated by a controller associated with the PCBA 112 and operatively connected to the pump 16, in a non-continuous pattern or duty cycle pattern. The system 10 may include a memory (integrated within PCBA 112) storing a first pump operation profile that defines operation of the pump 16, such as the non-continuous pattern, so as to activate the pump 16. In one or more versions, the first pump operation profile may include a predetermined pump on-time and pump off-time, effectively creating a “flush and hold” cycle that allows the medium in the thermal exchange pad 14 to reach thermal equilibrium with the wearer before being replaced.

In an embodiment, the memory stores a plurality of pump operation profiles that may include the first pump operation profile, from which the wearer allows for the selection of the desired profile directly or indirectly. For example, a second pump operation profile of the plurality of pump operation profiles may define continuous pump activation for maximum thermal transfer. A third pump operation profile may include a specific duty-cycle pattern optimized for battery conservation. The selection of the profiles may be achieved through a user interface or a connected application communicating with the PCBA 112.

In an embodiment, the system 10 may include one or more temperature sensors to detect any or a combination of temperatures associated with the system 10, the wearer of the system 10, and the operating environment. Based at least in part on these detected temperatures, the controller may regulates operation of the pump or volumetric flow, such as by adjusting a duty cycle of the pump 16, in an example pump off time and pump on time may also be known as the duty cycle. As shown in FIG. 3, the one or more temperature sensors are positioned to detect the temperature of the heat-transfer liquid entering (via inlet medium temperature sensor 52) and/or exiting (via outlet medium temperature sensor 54) the thermal exchange pad 14. Additionally, sensors may be positioned to detect temperature associated with the reservoir 12 and temperature associated with the pump 16.

In an embodiment, the one or more temperature sensors may detect the thermal exchange pad 14 at predetermined locations, monitoring the temperature of the drinking reservoir (if present), monitoring the temperature of the fluid reservoir 12, monitoring the temperature of the medium entering or exiting the pump 16, and/or monitoring the environmental temperature. Furthermore, the system 10 may be configured to monitor a skin temperature of the wearer, where all of these temperature inputs, whether utilized individually or in collaboration with one another, inform the controller to dynamically adjust the duty cycle performance of the pump 16 to optimize thermal regulation.

In an embodiment, the controller may regulate a volumetric flow of the heat-transfer liquid through at least a portion of the thermal exchange pad 14 to precisely control the thermal transfer between the wearer and the heat-transfer liquid. The controller may be configured to operate the pump 16 in various modes to achieve the regulation, including a non-continuous pattern, an intermittent mode, or a substantially continuous mode during use. Furthermore, the controller may vary a flow rate of the pump 16 (e.g., via voltage modulation) while maintaining continuous operation to adapt to changing thermal loads. To provide additional flow management flexibility, the system 10 may include a bypass conduit configured to selectively divert a portion of the heat-transfer liquid around at least a portion of the thermal exchange pad 14, or the system 10 may regulate thermal transfer by modulating hydraulic resistance (e.g., via variable valves or restrictors) within the thermal exchange pad 14 or generally within the closed loop.

In an example, the controller may be configured to implement a specific duty cycle logic where the pump-on time may be defined as a substantially fixed duration required to volumetrically flush the thermal exchange pad 14 with new heat-transfer medium (e.g., a 150 mL flush). During a cooling scenario, after this flush occurs, the pump 16 enters the pump off time or dwell period to allow for thermal equilibration and heat transfer to occur between the user and the medium now stationary within the thermal exchange pad 14. As the temperature of the medium in the reservoir 12 begins to rise (e.g., as ice melts and the water warms to 55° F.), the controller does not increase the on-time; rather, the controller reduces the off-time to minimize the dwell period between flushes. For example, the system 10 may initially operate with a cycle of 15 seconds on and 45 seconds off, and as the medium warms, the controller adjusts the cycle to 15 seconds on and 40 seconds off. This reduction in the off-time continues as the medium temperature rises, potentially progressing until the pump 16 operates continuously, which indicates to the user or the system that the thermal capacity of the reservoir 12 is depleted and requires recharging with ice or cooler water.

In an embodiment, the controller may execute specific control logic based on thermal feedback. For instance, the controller may increase the frequency of pump activation as the temperature of the heat-transfer liquid increases (e.g., to flush heat away during cooling mode). Conversely, the controller may increase the frequency of pump activation as the temperature of the heat-transfer liquid decreases (e.g., to replenish warm fluid during heating mode). To ensure safety and efficiency, the controller may decrease the frequency of pump activation when the temperature of the heat-transfer liquid is below a first predetermined threshold (preventing over-cooling) or when the temperature is above a second predetermined threshold (preventing over-heating). Furthermore, the controller may automatically adjust the duty cycle based on a physiological thermal model stored in the memory, thereby optimizing the system 10 for the specific metabolic needs of the wearer.

In one or more versions, the PCBA 112 provides a smart feedback and control for wearable thermal management system 10. Inlet medium temperature sensor 52 and an outlet medium temperature sensor 54 send temperature data/feedback to PCB 112. In one or more versions, system 10 may include other sensor types besides inlet medium temperature sensor 52 and an outlet medium temperature sensor 54 to provide additional smart feedback and control information. These other sensors may include a temperature sensor that detects the temperature of the user of system 10, a temperature sensor that detects the ambient temperature in the air surrounding the user of system 10, and/or an accelerometer to track the relative activity level of the user of system 10. In one or more versions, the smart feedback and control information may also be sent to other smart devices utilized by the user of system 10, and the data collected by these other smart devices may also be utilized by PCBA 112 to further hone the parameters of system 10.

The PCBA 112 may utilize this feedback to determine the effective working temperature of the user utilizing system 10. This working temperature helps achieve an optimal thermoregulation effect on the user with respect to the temperature of the user and the environment the user is operating in based on physiological thermal models that are built into the PCBA 112. This allows for optimal cooling/heating potential efficiency and power consumption of the power source 110 by promoting a safety function whereby the user cannot be over-regulated (cooled/warmed) via temperature monitoring and control of system 10. This feedback can also be utilized to indicate the status of the power source 110 and the temperature of the medium within reservoir 12, allowing the PCBA 112 to know when a total flush of medium within the reservoir 12 may be needed.

The smart feedback and control discussed above monitors and receives feedback at key points within system 10 which may result in the facilitation of automatic control of system 10 to reach an effective working temperature of the working fluid. Getting to an effective working temperature may achieve an optimal thermoregulation effect on the use of system 10 with respect to the temperature of the user based on physiological thermal models; may optimize efficiencies of system 10 and the overall power consumption of power source 110; may promote a safety function whereby the user cannot be over-regulated (cooled/warmed) via temperature monitoring and an automatic shutoff feature; and may indicate maintenance requirements of system 10, such as status of power source 110 and/or if the working fluid needs recharged. The smart feedback and control monitors and receives feedback at key points within system 10 may also result in the facilitation of self-monitoring or remote monitoring of the thermal-physiological state of the user of system 10 which may be achieved by the wired or wireless interconnection of system 10 to an interface or application that may allow for user input or review of the status of system 10 on a remote display such as within a mobile application; or through the creation of alerts or alarms that notify the user of system 10 or a remote monitor of system 10 of performance, activity, health status, and/or the temperature of the user, may initiate emergency care, and/or may facilitate the recognition of patterns of the user of system 10. The smart feedback and control monitors and receives feedback at key points within system 10, which may also allow for manual user input that permits real-time customization of the comfort feedback. Finally, the smart feedback and control monitors and receives feedback at key points within system 10 may allow for pre-programmed duty cycle inputs that flushes new working fluid into the thermal exchange pad 14 from the reservoir 12 while taking advantage of the equilibration that occurs during the thermoregulatory exchange.

In one or more versions, the PCBA 112 may be connected, either wirelessly or hard-wiring, to an interface or application that allows for user input on the use of system 10 as well as the remote display of the effective working temperature of the user as discussed above. In one or more versions, the interface or application can create alerts/alarms that notify the user of system 10 or others monitoring the status of the user that could indicate user performance, activity, health status, temperature (both ambient and core), remaining regulation capacity time, human metabolic rate, and other similar statuses of the user.

In one or more versions, system 10, namely the PCBA 112, may be pre-programmed with a duty-cycle input that flushes new working medium from the reservoir 12 into the thermal exchange pad 14. In one or more versions, the duty-cycle takes advantage of the equilibration of medium within the reservoir 12 for thermoregulatory exchange. In one or more versions, the duty-cycle may leverage voltage adjustments to the pump 16 that increase or decrease the flow rate based on user needs for thermal exchange and/or on/off cycle timing to address increased/decreased need for thermal exchange.

If the optional drinking tube port or medium distribution device is present, it may also be contemplated that system 10 can track fluid, calorie, and/or electrolyte intake for the user of system 10. This information may also be fed into the smart feedback and control. If calorie and electrolyte intake is being tracked, system 10 may also require an additional filtration system.

In an embodiment, the system 10 may include a flow control element operatively coupled to the pump 16 and positioned within the closed loop to provide precise hydraulic regulation. The controller is configured to regulate a flow rate of the heat-transfer liquid through the thermal exchange pad 14 by modulating the flow control element independently of the pump 16 on/off operation. For example, while the pump 16 maintains a constant discharge pressure or operational state, the flow control element, which may be a proportional valve, a variable flow restrictor, or a bypass valve, can be adjusted by the controller 112 to vary a volumetric throughput entering the thermal exchange pad 14, thereby allowing for fine-tuned thermal management without necessitating the complete cessation of pump operation.

In another embodiment, the wearable thermal management system 10 may include the reservoir 12 holding the heat-transfer liquid, the thermal exchange pad 14 positioned adjacent the wearer, and the pump 16 fluidly coupled to the reservoir 12 and the thermal exchange pad 14 to circulate the heat-transfer liquid through a closed loop. The system 10 further includes a non-electronic fluid circulation mechanism operatively coupled to the pump 16 or the closed loop to regulate volumetric flow of the heat-transfer liquid without digital or programmable electronic control. In one or more versions, the fluid circulation mechanism may include any or a combination of a cam configured to intermittently depress an actuator of the pump 16, a manual crank, a squeeze bulb, and a self-pumping mechanism that activates upon movement of the wearer. Such mechanical arrangements allow for the regulation of fluid flow without reliance on digital controllers.

In an embodiment, the fluid circulation mechanism utilizes mechanical timing or analog thermal feedback. For instance, the fluid circulation mechanism may include a spring-loaded escapement that releases periodically to activate the pump 16. Alternatively, the mechanism may include a bimetallic switch that interrupts electrical power based on temperature, providing automatic regulation based on the thermal state of the system 10. In other versions, the fluid circulation mechanism may include an analog RC timing circuit configured to intermittently energize the pump 16, or an electromechanical relay driven by a periodic timing signal.

In an embodiment, the system 10 may be integrated into the backpack 70 so as to route tubing 20 and wiring internally within the backpack 70 structure. The pump 16 may be any or a combination of a diaphragm pump, a peristaltic pump, and a centrifugal pump. The integration of the system 10 is not limited to a backpack; the system 10 may be adapted into various other wearable configurations, including a chest-mounted rig for conditions such as menopause symptom management or a waist pack for lower back pain relief. Furthermore, the system's form factor and utility extend to non-human applications, such as vest systems for canines or equines, or thermal regulation covers for sensitive equipment, effectively warming or cooling a target subject to maintain the subject within an acceptable temperature range. The non-continuous pattern or duty cycle generated by the non-electronic fluid circulation mechanism is selected so as to maintain a substantially constant thermal sensation at the wearer, thereby optimizing the heat transfer efficiency between the thermal exchange pad 14 and the wearer.

In another embodiment, the wearable thermal management system 10 may include a hydration reservoir (corresponding to the main volume of bladder 12) configured to hold a drinkable liquid and operatively coupled with a drinking tube (connectable via port 18d), and a thermoregulation reservoir (corresponding to distribution manifold pocket 24) configured to hold a heat-transfer liquid. The thermoregulation reservoir 24 may be operatively coupled to the pump 16 that circulates the heat-transfer liquid through the thermal exchange pad 14 positioned adjacent to a wearer, forming a closed thermal loop. The hydration reservoir 12 and the thermoregulation reservoir 24 are arranged to facilitate thermal energy transfer through a thermal interface (e.g., the shared walls or proximity within bladder 12), thereby enabling the thermoregulation reservoir 24 to absorb heat or cold from the hydration reservoir 12. In some configurations, the hydration reservoir and the thermoregulation reservoir form partitioned sections of a single main reservoir 12, while in others they are distinct sections operatively coupled with each other.

In an embodiment, the system 10 may include an input/output connector (such as connector 28a or 28b) operatively coupled with the pump 16, and a distribution manifold connector (such as connector 28c or 28e) operatively coupled with the pump 16 on one end and the thermal exchange pad 14 on the other end. The thermal exchange pad 14 is configured to act as a cooling pad if the heat-transfer liquid is cold, and as a heating pad if the heat-transfer liquid is hot or warm. To optimize thermal regulation, as shown in FIG. 3, the thermal exchange pad 14 includes an inlet medium temperature sensor 52 and/or an outlet medium temperature sensor 54. These sensors 52, 54 track the temperature of the heat-transfer liquid entering and exiting the thermal exchange pad 14. Based on the temperature data, the system 10 determines the amount of heat extracted and controls the amount of time for which the system 10 cycles the heat-transfer liquid.

In an embodiment, the system 10 may include one or more temperature sensors operatively coupled with any or a combination of the thermoregulation reservoir 24, the pump 16, the thermal exchange pad 14, and the wearer, providing additional data to control the liquid cycling. Structurally, as shown in FIGS. 3 and 10, the thermal exchange pad 14 may include the first layer 58a, the second layer 58b, and the baffle 56 running between the layers 58a, 58b to keep them in contact with each other. The entire system 10 may be operatively coupled with a backpack 70 (shown in FIG. 5), allowing for the internal routing of components such as tubing 20 while the user wears the system 10.

In another embodiment, the system 10 may eliminate the need for an electrical pump by utilizing alternative motive forces to circulate the heat-transfer liquid through the closed loop. For instance, gravity-driven flow may be achieved by positioning the reservoir at an elevation higher than the thermal exchange pad, utilizing a manual valve to regulate the rate at which the liquid descends into the pad. Alternatively, motion-driven flow utilizes the kinetic energy of the wearer; as the wearer walks or runs, the rhythmic vertical oscillation of the backpack 70 or the physical compression of a specific pump mechanism against the body acts to mechanically drive the fluid. The system 10 may also employ manual mechanisms, such as a squeeze bulb accessible on a shoulder strap or a hand-crank, allowing the user to manually prime the system 10 or generate intermittent flushes of the medium on demand. These non-electric configurations effectively reduce the system's acoustic signature and weight while removing dependence on battery power.

The present disclosure provides a robust wearable thermal management solution that significantly enhances user comfort and safety by effectively regulating body temperature through cooling or warming capabilities. A primary advantage lies in its efficient use of resources; by utilizing a single reservoir for both hydration and thermal exchange, the system 10 minimizes the weight and bulk carried by the user compared to systems requiring separate coolant supplies. Operational efficiency is further maximized through a smart controller that executes non-continuous pump duty cycles, leveraging thermal equilibration to extend battery life and optimize heat extraction. Additionally, the integration of a comprehensive sensor network ensures user safety by automatically adjusting performance to prevent hazardous over-cooling or over-heating, while the system's low-profile, baffled design ensures seamless compatibility with backpacks and tactical equipment without restricting mobility.

The following examples relate to various non-exhaustive ways in which the teachings herein may be combined or applied. It should be understood that the following examples are not intended to restrict the coverage of any claims that may be presented at any time in this application or in subsequent filings of this application. No disclaimer is intended. The following examples are being provided for nothing more than merely illustrative purposes. It is contemplated that the various teachings herein may be arranged and applied in numerous other ways. It is also contemplated that some variations may omit certain features referred to in the below examples. Therefore, none of the aspects or features referred to below should be deemed critical unless otherwise explicitly indicated as such at a later date by the inventors or by a successor in interest to the inventors. If any claims are presented in this application or in subsequent filings related to this application that include additional features beyond those referred to below, those additional features shall not be presumed to have been added for any reason relating to patentability.

### EXAMPLE 1

It should be understood that any of the versions of thermal management systems described herein may include various other features in addition to or in lieu of those described above. By way of example only, any of the thermal management systems described herein may also include one or more of the various features disclosed in any of the various references that are incorporated by reference herein. It should also be understood that the teachings herein may be readily applied to any of the thermal management systems described in any of the other references cited herein, such that the teachings herein may be readily combined with the teachings of any of the references cited herein in numerous ways. Moreover, those of ordinary skill in the art will recognize that various teachings herein may be readily applied to other versions of thermal management systems outside of the versions shown in the drawings. Other types of thermal management systems into which the teachings herein may be incorporated will be apparent to those of ordinary skill in the art.

It should be appreciated that any patent, publication, or other disclosure material, in whole or in part, that is said to be incorporated by reference herein is incorporated herein only to the extent that the incorporated material does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

Having shown and described various versions of the present invention, further adaptations of the methods and systems described herein may be accomplished by appropriate modifications by one of ordinary skill in the art without departing from the scope of the present invention. Several such potential modifications have been mentioned, and others will be apparent to those skilled in the art. For instance, the examples, embodiments, geometrics, materials, dimensions, ratios, steps, and the like discussed above are illustrative and are not required. Accordingly, the scope of the present disclosure should be considered in terms of the following claims and is understood not to be limited to the details of structure and operation shown and described in the specification and drawings.

We claim:

1. A wearable thermal management system comprising:
a reservoir holding a heat-transfer liquid;
a thermal exchange pad positioned adjacent a wearer;
a pump fluidly coupled to the reservoir and the thermal exchange pad to circulate the heat-transfer liquid through a closed loop; and
a controller operatively coupled to the pump, wherein the controller regulates volumetric flow of the heat-transfer liquid through at least a portion of the thermal exchange pad to control thermal transfer between the wearer and the heat-transfer liquid.

2. The system of claim 1, wherein the controller operates the pump in at least one of: a substantially continuous mode, an intermittent mode, or a variable flow rate continuous mode.

3. The system of claim 1, further comprising a bypass conduit configured to selectively divert a portion of the heat-transfer liquid around at least a portion of the thermal exchange pad.

4. The system of claim 1, wherein thermal transfer is further regulated by modulating hydraulic resistance within the thermal exchange pad or within the closed loop.

5. The system of claim 1, wherein the system further comprises a memory storing a first pump operation profile that defines operation of the pump, and wherein the first pump operation profile comprises a predetermined pump on-time and pump off-time, and wherein the memory stores a plurality of pump operation profiles comprising said first pump operation profile, from which the wearer selects directly or indirectly the first pump operation profile, and wherein a second pump operation profile of the plurality of pump operation profiles defines continuous pump activation, and wherein a third pump operation profile of the plurality of pump operation profiles comprises a duty-cycle pattern.

6. The system of claim 1, wherein the system further comprises one or more temperature sensors to detect any or a combination of temperatures associated with the system, wearer of said system, and operating environment, and wherein the controller regulates operation of the pump or volumetric flow based at least in part on the detected temperatures, and wherein the one or more temperature sensors are positioned to detect the temperature of any or a combination of heat-transfer liquid entering and/or exiting the thermal exchange pad, temperature associated with the reservoir, and temperature associated with the pump.

7. The system of claim 6, wherein the controller, based on configuration, increases frequency of pump activation as the temperature of the heat-transfer liquid increases or decreases.

8. The system of claim 6, wherein the controller decreases frequency of pump activation when the temperature of the heat-transfer liquid is below a first predetermined threshold, and wherein the controller decreases frequency of pump activation when the temperature of the heat-transfer liquid is above a second predetermined threshold.

9. The system of claim 6, wherein the controller automatically adjusts the volumetric flow based on a thermal model stored in a memory, and wherein the system further comprises a flow control element operatively coupled to the pump or positioned within the closed loop, and wherein the controller regulates a flow rate of the heat-transfer liquid through the thermal exchange pad by modulating the flow control element independently of pump on/off operation.

10. A wearable thermal management system comprising:
a reservoir holding a heat-transfer liquid;
a thermal exchange pad positioned adjacent a wearer;
a pump fluidly coupled to the reservoir and the thermal exchange pad to circulate the heat-transfer liquid through a closed loop; and
a non-electronic fluid circulation mechanism operatively coupled to the pump or the closed loop to regulate volumetric flow of the heat-transfer liquid without digital or programmable electronic control.

11. The system of claim 10, wherein the fluid circulation mechanism comprises any or a combination of cam configured to intermittently depress an actuator of the pump, manual crank, squeeze bulb, and self-pumping upon movement of the wearer.

12. The system of claim 10, wherein the fluid circulation mechanism comprises any or a combination of a spring-loaded escapement that releases periodically to activate the pump, a bimetallic switch that interrupts electrical power based on temperature, an analog RC timing circuit configured to intermittently energize the pump, and an electromechanical relay driven by a periodic timing signal.

13. The system of claims 10, wherein the system is integrated into a backpack so as to route tubing and wiring internally, and wherein the pump is any or a combination of a diaphragm pump, a peristaltic pump, and a centrifugal pump, and wherein operation of the system is selected so as to maintain a substantially constant thermal sensation at the wearer.

14. A wearable thermal management system comprising:
a hydration reservoir to hold a drinkable liquid, said hydration reservoir being operatively coupled with a drinking tube;
a thermoregulation reservoir to hold a heat-transfer liquid, said thermoregulation reservoir being operatively coupled to a pump that circulates said heat-transfer liquid through a thermal exchange pad positioned adjacent to a wearer so as to form a closed thermal loop, wherein hydration reservoir and the thermoregulation reservoir are arranged so as to allow thermal energy transfer through a thermal interface so as to enable the thermoregulation reservoir to absorb heat or cold from the hydration reservoir.

15. The system of claim 14, wherein the hydration reservoir and the thermoregulation reservoir form partitioned sections of a single main reservoir, and wherein the hydration reservoir and the thermoregulation reservoir are sections that are operatively coupled with each other.

16. The system of claim 14, wherein the system comprises:
an input/output connector operatively coupled with the pump; and
a distribution manifold connector operatively coupled with the pump on one end and the thermal exchange pad on the other end.

17. The system of claim 14, wherein the thermal exchange pad acts as a cooling pad if the heat-transfer liquid is cold, and acts as a heating pad if the heat-transfer liquid is hot/warm.

18. The system of claim 14, wherein the thermal exchange pad comprises an inlet medium temperature sensor and/or an outlet medium temperature sensor to measure a temperature of the heat-transfer liquid entering and exiting the thermal exchange pad, based on which an amount of heat extracted by the thermal exchange pad is determined, and wherein circulation of the heat-transfer liquid is regulated by controlling volumetric flow through the thermal exchange pad.

19. The system of claim 14, wherein the system comprises one or more temperature sensors operatively coupled with any or a combination of the thermoregulation reservoir, the pump, the thermal exchange pad, and the wearer, based on which, amount of heat extracted is determined and circulation of the heat-transfer liquid is regulated by controlling volumetric flow.

20. The system of claim 14, wherein the thermal exchange pad comprises a first layer and a second layer, and a baffle that runs between the said layers to keep the layers in contact with each other, and wherein the system comprises a backpack operatively coupled with the thermal exchange pad.
